(19) United States
(12) Patent Application Publication
(10) Pub. No.: US 2007/0219432 A1
(43) Pub. Date: Sep. 20, 2007

(54) Method and Apparatus for Automatic Detection of Meter Connection and Transfer of Data

(76) Inventor: Brian C. Thompson, Osceola, IN (US)

Correspondence Address: NIXON PEABODY LLP, 161 N. CLARK STREET, 48TH FLOOR, CHICAGO, IL, 60601, US

(21) Appl. No.: 11/596,246

(22) PCT Filed: May 13, 2005
(86) PCT No.: PCT/US05/16897

§ 371 Date: Nov. 13, 2006

Related U.S. Application Data
(60) Provisional application No. 60/571,096, filed on May 14, 2004.

(51) Int. Cl. A61B 5/00

(52) U.S. Cl. 600/300

(57) ABSTRACT

A method and apparatus are provided for implementing data management with a data collection computer system to aid analysis and treatment. A serial port is monitored to detect the connection of a meter. When a meter connection is identified, patient data is automatically downloaded from the meter to the data collection computer system. Then the patient data or one or more selected reports generated from the patient data are printed. The patient data is downloaded from the meter and printed, with no user intervention required. Reports to be generated and printed are selected in a setup mode and stored. After the patient data is downloaded from the meter, communication with the meter is continued until either the meter is turned off, the cable is disconnected, or the meter automatically shuts itself off after a specific amount of inactivity.

### CROSS-REFERENCE TO RELATED APPLICATIONS

This application claims priority to Application No. 60/571,096, filed May 14, 2004, which is incorporated by reference in its entirety.

### FIELD OF THE INVENTION

The present invention relates generally to the biosensors, and more particularly, relates to a method and apparatus for implementing automatic detection of a meter connection, such as, a blood glucose meter, and automatic transfer of data.

### DESCRIPTION OF THE RELATED ART

The quantitative determination of analytes in body fluids is of great importance in the diagnoses and maintenance of certain physiological abnormalities. For example lactate, cholesterol and bilirubin should be monitored in certain individuals. In particular, the determination of glucose in body fluids is of great importance to diabetic individuals who must frequently check the level of glucose in their body fluids as a means of regulating the glucose intake in their diets. While the remainder of the disclosure herein will be directed towards the determination of glucose, it is to be understood that the procedure and apparatus of this invention can be used with other diagnostic systems.

Home glucose monitoring by diabetics is becoming increasingly routine in modern day diabetes management. Historically patients were required to maintain hand written paper log books for manually recording glucose readings and other relevant information. More specifically, patients measured their blood glucose at scheduled times, and recorded this information in a personal log book.

Known diagnostic systems, such as, blood glucose systems include a biosensor used to calculate the actual glucose value based on a measured output and the known reactivity of the reagent sensing element used to perform the test. The test results typically are displayed to the user and stored in a memory in the blood glucose meter. In some known systems, the multiple stored values from the blood glucose meter are periodically transferred to a separate computer, for example to enable analysis by a doctor for the blood glucose monitor user.

While the introduction of glucose meters with various memory functions has greatly simplified the data recording process and increased the reliability of stored data, the large amounts of recorded data have made the interpretation task complicated. It is also possible with present day devices for patients to record other clinically relevant data such as diet and exercise factors, and life style information. All such stored data can conveniently be transferred to a physician's office, typically via a communications link such as a direct meter cable connection or an acoustic modem line, where it can be reviewed in printed or displayed form for making appropriate treatment recommendations.

Many traditional approaches to automated analysis of diabetes data provide a relatively superficial analysis and an assortment of graphical displays based upon certain predefined statistical calculations. However, the time consuming and complicated synthesis and interpretation of clinical implications associated with the processed data still need to be performed by the reviewing physician, and significant interaction is still required on behalf of the physician.

U.S. Pat. No. 5,251,126 issued Oct. 5, 1993 to Kahn et al., and assigned to the present assignee discloses an automated diabetes data interpretation method referred to as the “IDDI” system, that combines symbolic and numeric computing approaches in order to identify and highlight key clinical findings in the patient's self recorded diabetes data. The patient data, including blood glucose levels and insulin dosage levels, recorded by a diabetic patient over a period of time by means of a glucose meter or the like, is initially downloaded into a central processing system such as a personal computer. The accepted diabetes data is processed to (a) identify insulin dosage regimens corresponding to predefined significant changes in insulin dosage that are found to be sustained for at least a predefined segment of the overall data collection period, (b) identify statistically significant changes in blood glucose levels resulting across adjacent ones of the identified insulin regimen periods, and (c) identify clinically significant changes in blood glucose levels from within the identified statistically significant glucose level changes. The results of the diabetes data processing are generated in the form of a comprehensive yet easily understandable data interpretation report highlighting the processing results, including details pertaining to the identified insulin regimens and the associated clinically significant changes in glucose levels.

Multiple commercially available clinical analyzers are available for patient use. Due to differences between various commercially available clinical analyzers, a health care professional (HCP) must have compatible software to run, or may require the patient to be present in the HCP's office if the patient does not have the same or similar program at home. The HCP must run the program, switch cables to match the meter, and maintain both hardware and software. Such chores tend to be time consuming and inefficient.

A need exists for an improved method and apparatus for implementing data management to aid analysis and treatment by the patient's doctor or HCP and to minimize time required, for example, in running software, switching cables, and downloading meters.

### SUMMARY OF THE INVENTION

Important aspects of the present invention are to provide a new and improved method and apparatus for implementing data management to aid analysis and treatment including automatic detection of a meter connection, such as, a blood glucose meter and automatic transfer of data to aid analysis and treatment; to provide such method and apparatus that eliminates or minimizes the need for user interaction; and to provide such method and apparatus that overcome some disadvantages of prior art arrangements.

In brief, a method and apparatus are provided for implementing data management with a data collection computer system to aid analysis and treatment. A serial port is monitored to detect the connection of a meter. When a meter connection is identified, patient data is automatically downloaded from the meter to the data collection computer system. Then the patient data or one or more selected reports generated from the patient data are printed.

In accordance with features of the invention, the patient data is downloaded from the meter and printed, with no user intervention required. Reports to be generated and printed are selected in a setup mode and stored. After the patient data is downloaded from the meter, communication with the meter is continued until either the meter is turned off, the cable is disconnected, or the meter automatically shuts itself off after a specific amount of inactivity.

### BRIEF DESCRIPTION OF THE DRAWINGS

The present invention together with the above and other objects and advantages may best be understood from the following detailed description of the preferred embodiments of the invention illustrated in the drawings, wherein:

FIG. 1A illustrates an exemplary data collection computer system for implementing automatic detection of a meter connection and automatic transfer of data in accordance with the present invention;

FIG. 1B is a logical block diagram representation of the data collection computer system of FIG. 1A for implementing automatic detection of a meter connection and automatic transfer of data in accordance with the present invention; and

FIGS. 2 and 3 are flow charts respectively illustrating exemplary steps performed by the data collection computer system of FIGS. 1A and 1B in accordance with the automatic meter detection and data transfer methods in accordance with the present invention.

### DETAILED DESCRIPTION OF THE PREFERRED EMBODIMENTS

In accordance with features of the invention, a software method of a data collection computer system monitors at least one serial port for the presence of a blood glucose meter. Upon detection of an attached blood glucose meter, the software downloads the data from the meter, prints the data and predefined reports, and waits for the attached meter to be turned off or removed from an attached cable. This last step is important otherwise, the same meter will be detected again and dumped and printed again. All this happens without pressing any keys on the data collection device. This method represents a new level in user friendliness.

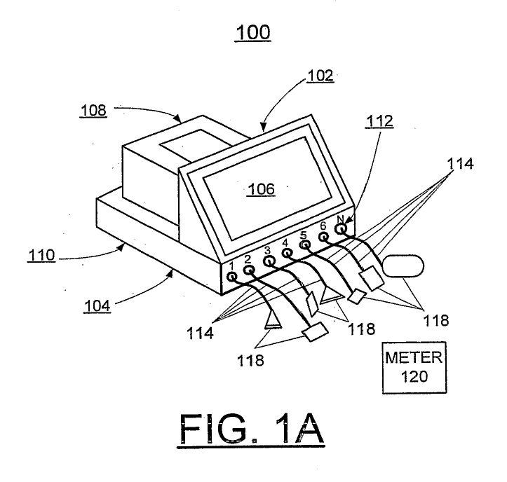

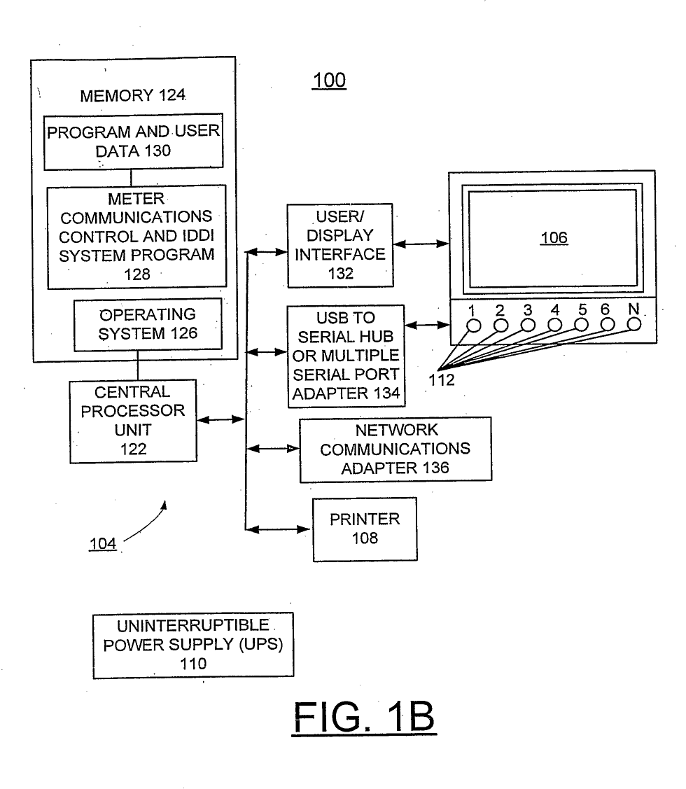

Having reference now to the drawings, in FIGS. 1A and 1B, there is illustrated an exemplary computer system designated as a whole by the reference character 100 and arranged in accordance with principles of the present invention. Data collection computer system 100 includes a housing generally designated by reference character 102 containing a computer 104, a display touch screen 106, a printer 108, and an optional uninterruptible power supply 110. Data collection computer system 100 is a unitary system typically located in an office of a health care professional (HCP). Data collection computer system 100 is arranged for use by patients without requiring assistance from any HCP.

As shown, data collection computer system 100 includes a plurality of ports 1-N, 112, each receiving a respective cable 114. An associated connector 118 is provided with each of the plurality of cable 1-N, 114 for electrically connecting with a particular meter 120. Each of the multiple connectors 118 is arranged for use with a particular one of multiple meter types.

The meter 120, such as a biosensor or glucose meter 120 is used by a patient and periodically receives and processes a user sample from the patient, then stores or records the measured blood glucose (BG) levels. The meter 120 is attached to its specific cable 1-N, 114 via the associated connector 118 mating with the meter. Some blood glucose meters must be turned on in order to communicate with the data collection computer system 100.

Referring also to FIG. 1B, computer 104 includes a central processor unit (CPU) 122 together with an associated memory 124. Computer 104 includes an operating system 126, a meter communications control and IDDI system program 128 of the preferred embodiment, and program and user data 130 of the preferred embodiment resident in memory 124. Computer 104 includes a user/display interface 132 that couples the display touch screen 106 to the CPU 122, and a USB to serial hub or multiple serial port adapter 134 that couples an attached meter 120 to the CPU 122. Computer 104 includes a network communications adapter 136 for connection, for example, to another computer (not shown) in the doctor's office.

Data collection computer system 100 is shown in simplified form sufficient for understanding the present invention. The illustrated computer test system 100 is not intended to imply architectural or functional limitations. The present invention can be used with various hardware implementations and systems and various other internal hardware devices.

The meter communications control and IDDI system program 128 directs the data collection computer system 100 to automatically download patient data and print out data and reports in accordance with the preferred embodiment. The meter communications control and IDDI system program 128 includes the automated intelligent diabetes data interpretation (IDDI) software functions necessary to process, analyze and interpret the self recorded diabetes patient data and generate selected reports.

U.S. Pat. No. 5,251,126 issued Oct. 5, 1993 to Kahn et al., and assigned to the present assignee, discloses an IDDI system that advantageously included in the IDDI software functions of the meter communications control and IDDI system program 128 in the data collection computer system 100. The subject matter of the above identified U.S. Pat. No. 5,251,126 is incorporated herein by reference.

In accordance with features of the invention, the meter communications control and IDDI system program 128 attempts to communicate with a blood glucose meter 120 by utilizing commands that the blood glucose meter normally responds or acknowledges. Once a response is received, the program 128 knows that a meter is attached. The program 128 then proceeds to download the data without requiring a key press or any user entry to the data collection computer system 100. Once the data is downloaded, one or more printouts advantageously is made automatically without requiring a key press or any user entry to the data collection computer system 100. The printouts specifically requested are setup in a special setup mode of the program 128 and stored in the program and user data 130 in memory 124. Then the program 128 communicates with the meter 120 using commands that the blood glucose meter normally responds. The software will continue to communicate with the blood glucose meter until either the meter is turned off, the cable is disconnected, or the meter automatically shuts itself off after a specific amount of inactivity.

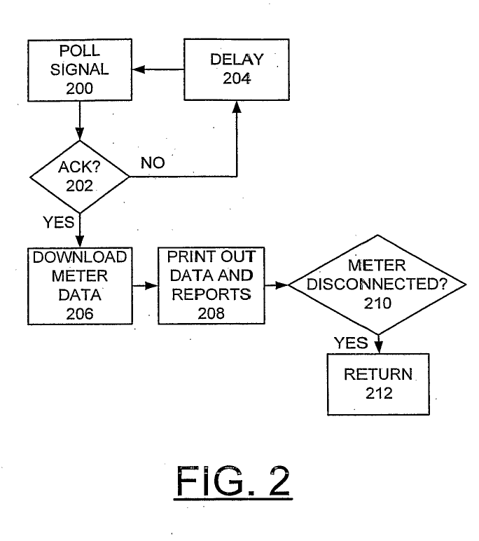

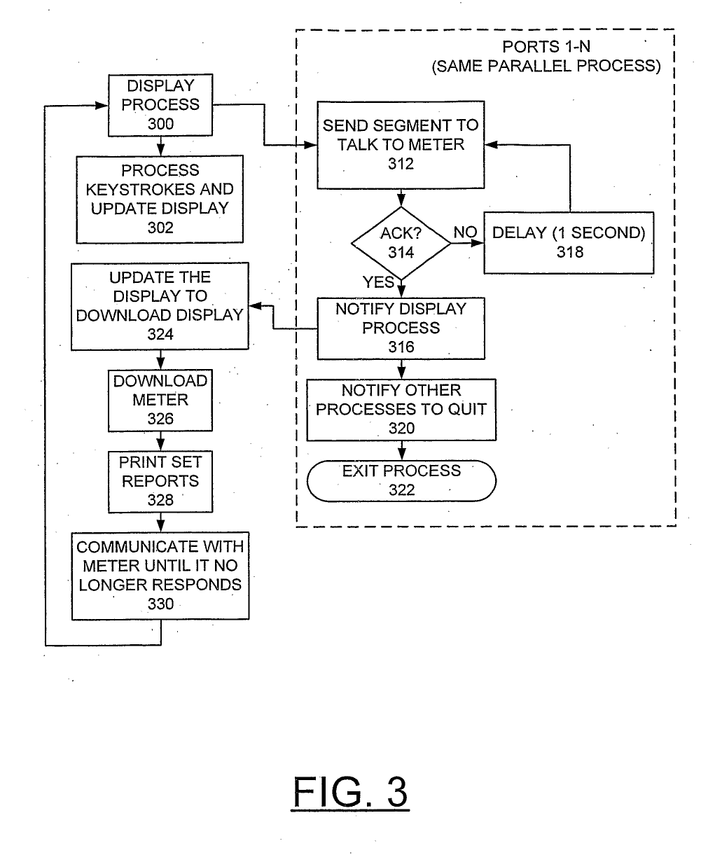

FIGS. 2 and 3 are flow charts respectively illustrating exemplary steps performed by the data collection computer system 100 of FIGS. 1A and 1B in accordance with the automatic meter detection and data transfer methods in accordance with the present invention.

Referring now to FIG. 2, the CPU 122 of the data collection computer system 100 sends a poll signal attempting to communicate with a blood glucose meter 120 as indicated in a block 200 and checks for an acknowledgement signal from the blood glucose meter 120 as indicated in a decision block 202. When an acknowledgement signal from the blood glucose meter 120 is not identified, a set delay is provided as indicated in a block 204 then another poll signal is sent at block 200. When an acknowledgement signal from the blood glucose meter 120 is identified, then the meter data is downloaded as indicated in a block 206. Then patient data and reports generated from the patient data are printed as indicated in a block 208. Checking for the meter being disconnected is performed as indicated in a decision block 210. When the meter is disconnected, then the sequential steps return as indicated in a block 212.

Referring now to FIG. 3, the CPU 122 of the data collection computer system 100 performs a display process for viewing by a patient as indicated in a block 300. User entries or keystrokes are processed and the display is updated responsive to the user entries as indicated in a block 302. Parallel identical processes are performed for each of the meter ports 1-N, as indicated in a plurality of blocks 312, 314, 316, 318, 320, and 322. A segment is sent to talk to meter 120 as indicated in a block 312. Checking for an acknowledgement signal from the blood glucose meter 120 as indicated in a decision block 314. When an acknowledgement signal from the blood glucose meter 120 is not identified, a delay is provided as indicated in a block 318. When an acknowledgement signal from the blood glucose meter 120 is identified, then the display is updated to a predefined download display as indicated in a block 324. Other processes are notified to halt or quit as indicated in a block 320. Then the process exits as indicated in a block 322. The meter data is downloaded as indicated in a block 326. Then patient data and reports generated from the patient data are printed as indicated in a block 328. Communications with the meter 120 is continued until the meter no longer responds as indicated in a block 330.

The software will attempt to communicate with a blood glucose meter by utilizing commands that the blood glucose meter normally responds to. Once a response is received, the software knows that a meter is attached. The software then can proceed to download the data without requiring a key press on the data collection device. Once the data is downloaded, the printout can be made automatically without requiring a key press on the data collection device. The printouts specifically requested are setup in a special setup mode in the software. Then the software will communicate with the meter using commands that the blood glucose meter normally responds to. The software will continue to communicate with the blood glucose meter until either the meter is turned off, the cable is disconnected, or the meter automatically shuts itself off after a specific amount of inactivity.

In brief summary, a primary difference from existing software arrangements is that the method of the invention is used to determine when a meter is present and to automatically download and print the data and selected reports without requiring any interaction from a user. Known existing software relies on the user to press a button to start the data transfer once the meter is prepared properly for data to be downloaded. Also, the method of the invention detects when the meter has been disconnected and avoids downloading the patient data more than once.

The downloaded patient data is processed by the data collection computer system 104 in accordance with the meter communications control and IDDI system program 128 in order to extract clinically meaningful information that is presented in a predefined report. The report is particularly adapted for convenient use by a physician toward arriving at meaningful or intelligent clinical and/or therapeutic decisions, and possibly can eliminate review by the physician of the raw data contained in the patient meter. It should be understood that the meter communications control and IDDI system program 128 requires no user intervention. The printed reports contain, for example, highlighted text, graphs, and tables, global comments, modal day analysis, modal week analysis, last two periods comparison, insulin dosage effects analysis, hypo and hyperglycemic episodes, rapid swing in glucose levels, and the like.

While the present invention has been described with reference to the details of the embodiments of the invention shown in the drawing, these details are not intended to limit the scope of the invention as claimed in the appended claims.

1. A method for implementing data management with a data collection computer system, said method comprising the acts of: monitoring a predefined port to detect the connection of a meter, responsive to a meter connection being identified; automatically downloading patient data from the meter to the data collection computer system;
and printing a selected one or more of the downloaded patient data and a selected report generated from the patient data.

2. The method for implementing data management of claim 1 wherein the act of monitoring a predefined port to detect the connection of a meter includes the acts of monitoring a predefined serial port of the data collection computer system for detecting the connection of a meter.

3. The method for implementing data management of claim 1 wherein the act of monitoring a predefined port to detect the connection of a meter includes the acts of sending a poll signal to the predefined port, and checking for an acknowledgement signal from a meter.

4. The method for implementing data management of claim 1 further includes the acts of processing said patient data and generating an intelligent diabetes data interpretation (IDDI) report.

5. The method for implementing data management of claim 1 wherein the acts of automatically downloading patient data and printing acts are performed with no user intervention required.

6. The method for implementing data management of claim 1 further includes the acts after the patient data is downloaded from the meter, of continuing communication with the meter until either the meter is turned off, the cable is disconnected, or the meter automatically shuts itself off after a specific amount of inactivity.

7. The method for implementing data management of claim 1 includes multiple processes performed in parallel for multiple predefined ports.

8. The method for implementing data management of claim 1 further includes the acts of displaying instructions for connecting a meter to one of multiple cables.

9. The method for implementing data management of claim 8 further includes the acts of identifying a user entry and displaying updated information.

10. The method for implementing data management of claim 1 further includes the acts of storing program and user data, said program and user data including one or more report selections stored during a setup mode of the data collection computer system.

11. An apparatus for implementing data management comprising:
a processor device adapted to monitor a predefined port to detect the connection of a meter, said processor responsive to a meter connection being identified, for automatically downloading patient data from the meter and for generating a selected report from the patient data; and
a printer coupled to said processor for printing a selected one or more of the downloaded patient data and said selected report generated from the patient data.

12. The apparatus for implementing data management of claim 11 further including a memory coupled to said processor device, said memory adapted to store a meter communications control and intelligent diabetes data interpretation (IDDI) system program.

13. The apparatus for implementing data management of claim 12 further including said memory storing program and user data, said meter communications control and intelligent diabetes data interpretation (IDDI) system program causing said processor device to perform the acts of identifying said selected report from said stored storing program and user data without user intervention being required.

14. The apparatus for implementing data management of claim 12 further including a display touch screen coupled to said processor device for receiving said report selection stored in said memory during a setup mode.

15. The apparatus for implementing data management of claim 14 further including an uninterruptible power supply coupled to said processor device and said printer

16. The apparatus for implementing data management of claim 15 further including a unitary housing containing said processor device, said printer, said display touch screen, and said uninterruptible power supply.

17. The apparatus for implementing data management of claim 12 further including a plurality of cables, each for connection to a meter; and wherein said meter communications control and intelligent diabetes data interpretation (IDDI) system program causes said processor device to perform the acts of displaying instructions for connecting a meter to one of said plurality of cables.

18. The apparatus for implementing data management of claim 12 wherein said meter communications control and intelligent diabetes data interpretation (IDDI) system program causes said processor device to perform the acts of identifying a user entry and displaying information responsive to said user entry.
